(19) United States
(12) Patent Application Publication
(10) Pub. No.: US 2017/0231968 A1
(43) Pub. Date: Aug. 17, 2017

(54) METHOD FOR RELIEF OF AND TREATMENT OF PRURITUS

(71) Applicant: PellePharm, Inc., Orinda, CA (US)

(72) Inventor: Ervin Epstein, Orinda, CA (US)

(21) Appl. No.: 15/429,006

(22) Filed: Feb. 9, 2017

Related U.S. Application Data
(60) Provisional application No. 62/293,898, filed on Feb. 11, 2016.

(51) Int. Cl. A61K 31/4355; A61K 9/12; A61K 9/06; A61K 9/00

(52) U.S. Cl. CPC A61K 31/4355; A61K 9/0014; A61K 9/122; A61K 9/06

(57) ABSTRACT

A method for treating pruritus by topically applying a composition comprising a hedgehog inhibitor compound is provided.

### CROSS-REFERENCE TO RELATED APPLICATIONS

This application claims the benefit of U.S. Provisional Application No. 62/293,898, filed Feb. 11, 2016, incorporated herein by reference in its entirety.

### TECHNICAL FIELD

The subject matter described herein relates to a method for treating pruritus by topically applying a composition comprising a hedgehog inhibitor compound.

### BACKGROUND

Pruritus, or itch, is a sensation that stimulates the desire or reflex to scratch, which can be either generalized or localized. It is a characteristic feature of many skin diseases and can be a sign of certain systemic diseases. Pruritus may be localized or generalized and can occur as an acute or chronic condition. Itching lasting more than 6 weeks is termed chronic pruritus.

Itching can be intractable and incapacitating, as well as a diagnostic and therapeutic challenge. The best understood mechanism of itching is the release of histamine in the skin leading to urticarial wheals and intense itching. On this belief, itching has traditionally been relieved by antihistamines. While antihistamine therapy is often effective, the sedation and drowsiness produced by antihistaminic agents limits their effectiveness. Yet, not all itching is relieved by antihistamines. For example, conditions such as Hodgkin's disease, mycosis fungoides (cutaneous malignacy) and severe jaundice produce intense itching unrelieved by antihistamines. Subjects with tumors or with lesions of the central nervous system can also report intractable pruritus. Administration of opioids in epidural anesthesia can also cause pruritus.

Therefore, there is a need for therapies to relieve itching, including itching that does not respond to topical or oral antihistamines. The method disclosed herein offers such a therapy, and is beneficial for relief in intractable cases of pruritus which heretofore have been difficult to relieve and/or treat.

### BRIEF SUMMARY

The following aspects and embodiments thereof described and illustrated below are meant to be exemplary and illustrative, not limiting in scope.

In one aspect, a method for treating pruritus in a mammalian subject is provided. The method comprises topically applying a composition comprising a hedgehog inhibitor in an amount effective for treatment of pruritus.

In another aspect, a method for relieving itching on mammalian skin of a subject is provided, where the method comprises topically applying a composition comprising a hedgehog inhibitor compound in an amount effective to relieve itching.

In one embodiment, the composition comprises a liquid, a semi-solid or a solid composition. In an exemplary embodiment, the composition is a foam, a suspension, an emulsion or an ointment.

In another embodiment, the composition is topically applied to the skin at the site of itch. In another embodiment, the composition is topically applied to the skin in an area peripheral or adjacent the site of itch.

In yet another embodiment, the composition is a semi-solid or solid composition and the method further comprises rubbing the composition into the skin.

In still another embodiment, the composition is topically applied to the skin periodically less than once per day. In other embodiments, the composition is topically applied to the skin periodically from 1-8 times per day.

The composition, is various embodiments, comprises between about 0.01-10 percent by weight of the hedgehog inhibitor.

In one embodiment, the hedgehog inhibitor is patidegib.

In various embodiments, the pruritus or itching is associated with a pruritic condition selected from atopic dermatitis, nervous dermatitis, contact dermatitis, seborrheic dermatitis, autosensitization dermatitis, caterpillar dermatitis, asteatosis, senile pruritus, insect sting or bite, photosensitive dermatosis, urticaria, prurigo, herpes, impetigo, eczema, tinea, lichen, psoriasis, scabies and acne vulgaris.

In yet other embodiments, the pruritus or itching is associated with an underlying medical condition. Exemplary, non-limiting underlying medical conditions are those with an origin comprising dermatologic origin, systemic disease origin, neurologic origin, or psychogenic origin.

In one embodiment, the subject has uremic pruritus or prurigo nodularis.

In another embodiment, the subject has pruritus or itching secondary to a systemic condition selected from an endocrine or metabolic disease, an infectious disease, pregnancy, or administration (oral, topical, parenteral, etc.) of a drug.

In exemplary embodiments, the endocrine or metabolic disease is selected from chronic renal failure, diabetes mellitus, hyperthyroidism, hypothyroidism, liver disease, malabsorption and perimenopausal pruritus.

In other embodiments, the infectious disease is selected from helminthosis, a viral infection (such as but not limited to an HIV infection, a herpes simplex viral infection, a herpex zoster viral infection) and parasitosis.

In still other embodiments, the subject is pregnant and suffers from pruritus gravidarum with or without cholestasis.

Additional embodiments of the present methods will be apparent from the following description, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

As used in this specification, the singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The compositions of the present disclosure can comprise, consist essentially of, or consist of, the components disclosed.

All percentages, parts and ratios are based upon the total weight of the topical compositions and all measurements made are at about 25° C., unless otherwise specified.

For any particular compound disclosed herein, any general or specific structure presented also encompasses all conformational isomers, regioisomers, and stereoisomers that can arise from a particular set of substituents, unless stated otherwise. Similarly, unless stated otherwise, the general or specific structure also encompasses all enantiomers, diastereomers, and other optical isomers whether in enantiomeric or racemic forms, as well as mixtures of stereoisomers, as would be recognized by a skilled artisan.

The phrase “pharmaceutically acceptable” is employed herein to refer to those compounds, salts, compositions, dosage forms, etc., which are-within the scope of sound medical judgment-suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

The term “treating” is used herein, for instance, in reference to methods of treating pruritus, and generally includes relief of, reducing the frequency of, delaying the progression of, curing or, and alleviating itching.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The terms “administer,” “administering” or “administration” as used herein refer to either directly administering a compound or pharmaceutically acceptable salt of the compound or a composition to a subject.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent across the stratum corneum.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used in this disclosure, the term “subject” includes, without limitation, a human or an animal. Exemplary animals include, but are not limited to, mammals such as mouse, rat, guinea pig, dog, cat, horse, cow, pig, monkey, chimpanzee, baboon, or rhesus monkey.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

### II. Method of Treating Pruritus

In a first aspect, a method of treating pruritus comprising topically administering an effective amount of a hedgehog inhibitor compound to a subject in need of such treatment. Below, compositions for topical application and hedgehog inhibitor compounds are described.

### A. Compositions for Topical Application

The method comprises topically applying a composition comprising a hedgehog inhibitor compound. The hedgehog inhibitor compound can be formulated with any pharmaceutically-acceptable carrier, and may be a liquid, semi-solid or solid composition. Pharmaceutical and cosmetic carriers or vehicles suitable for topical administration of the compositions to the skin or mucosa are known to those skilled in the art and the hedgehog inhibitor compound can be included in the carrier in an amount sufficient to provide a therapeutically useful effect in the treatment of itch. Examples of carriers and various compositions are provided in the following paragraphs.

In one embodiment, the composition comprising a hedgehog inhibitor compound is a liquid. Liquid dosage forms for topical administration include emulsions, solutions and suspensions containing diluents commonly used in the art, such as alcohols, glycols, oils, water and the like. The compositions may also include wetting agents, emulsifying and suspending agents.

The compositions may be in the form of solutions, suspensions, emulsions, ointments, lotions, gels, and the like. Emulsions of the form oil-in-water or water-in-oil are contemplated. Gels are formed by the entrapment of large amounts of aqueous or aqueous-alcoholic liquids in a network of polymers or of colloidal solid particles. Such polymers or colloids are typically present at concentrations of less than 10% w/w and are also referred to as gelling agents or thickening agents. Examples of suitable gelling agents include carboxymethyl cellulose, hydroxypropylmethyl cellulose, hydroxyethyl cellulose, methyl cellulose, sodium alginate, alginic acid, pectin, tragacanth, carrageen, agar, clays, aluminum silicate, carbomers, etc.

Creams and ointments may also be utilized. They are emulsions of oleaginous substances and water (i.e. the carrier). The cream may be a water-in-oil (w/o) in which an aqueous phase is dispersed in an oil phase, or an oil-in-water (o/w) which have an oil dispersed within an aqueous base. An ointment is also contemplated, and is typically more viscous than an oil-in-water cream. Traditional ointment bases (i.e. the carrier) include hydrocarbons (petrolatum, beeswax, etc.) vegetable oils, fatty alcohols (cholesterol, lanoilin, wool alcohol, stearyl alcohol, etc.) or silicones. Pastes are a type of ointment into which a high percentage of insoluble particulate solids have been added, up to 50% by weight. Insoluble solids such as starch, zinc oxide, calcium carbonate, or talc may be used.

Aerosols may also be utilized. The compound may be dissolved in a propellant and a co-solvent such ethanol, acetone, hexadecyl alcohol, etc. Foaming agents may be incorporated to produce a mousse.

Emollient or lubricating vehicles that help hydrate the skin can also be used. Examples of suitable bases or vehicles for preparing hydrating compositions for use with human skin are petrolatum, petrolatum plus volatile silicones, lanolin, cold cream (USP), and hydrophilic ointment (USP).

A wide variety of methods may be used for preparing the formulations described above. Broadly speaking, the formulations may be prepared by combining together the components of the formulation, as described herein, at a temperature and for a time sufficient to provide a pharmaceutically acceptable composition. The term “combining together”, as used herein, means that all of the components of the compositions may be combined and mixed together at about the same time. The term “combining together” also means that the various components may be combined in one or more sequences to provide the desired product. The formulation can be prepared on a weight/weight (w/w) or a weight/volume (w/v) basis depending upon the form of the final dosage form.

The compositions comprise a weight fraction of a hedgehog inhibitor compound, that may be dissolved, suspended, dispersed or otherwise mixed in a selected carrier or vehicle, at an effective concentration such that the pruritic condition is relieved or ameliorated. Compositions that are in solution form and intended for topical administration may contain an amount a hedgehog inhibitor compound between about 0.01% w/w to about 25% w/w, or between about 0.01-10% w/w, with the balance of the solution being water, a suitable organic solvent or other suitable solvent or buffer. Compositions that are formulated as solutions, emulsions, or suspensions can be applied to the skin, or can be formulated as an aerosol or foam and applied to the skin as a spray-on. The aerosol compositions typically contain from 25% to 80% w/w, preferably from 30% to 50% w/w, of a suitable propellant.

Compositions of solid forms intended for topical application can be formulated as stick-type compositions intended for application to the lips or other parts of the body. Such compositions contain an effective amount of a hedgehog inhibitor compound (including a pharmaceutically acceptable salt or ester thereof). The amount of the hedgehog inhibitor compound is typically from about 0.01% w/w to about 25% w/w or between about 0.01-10% w/w. The solid form of the composition may also contain from about 40% to 98% w/w, preferably from about 50% to 90% w/w, of carrier(s).

In addition, the compositions, and preparations containing the compositions, can also be coated on bandages, mixed with bioadhesives, or included in dressings. Thus, combinations of bandages, bioadhesives, dressings and other such materials and the compositions formulated as described herein are provided.

The compositions employed in the present methods relieve pruritus when applied to the skin. The composition can be administered topically to the affected area as needed to provide reduction in and/or relief from itching. Relief can be temporary or permanent, and can even be evident after a single dose of the composition.

The compositions may be packaged for use in a medical setting or for retail distribution directly to the consumer (i.e., an article of manufacture or kit). Such articles will be labeled and packaged in a manner advising the patient how to use the product for therapy. Such instructions will include the duration of treatment, dosing schedule, precautions, etc. These instructions may be in the form of pictures, written instructions, or a combination thereof. They may be printed on the side of the packaging, be an insert, or any other form of communication appropriate for the retail market.

### B. Hedgehog Inhibitor Compounds

Hedgehog inhibitor compounds contemplated for use include, for example, those described and disclosed in U.S. Pat. Nos. 7,230,004, 7,812,164; 8,669,365, U.S. Patent Application Publication No. 2008/0287420, U.S. Patent Application Publication No. 2008/0293755 and U.S. Patent Application Publication No. 2013/0109700, the entire disclosures of which are incorporated by reference herein. Examples of other suitable hedgehog inhibitors include those described in U.S. Patent Application Publication Nos. US 2002/0006931, US 2007/0021493 and US 2007/0060546, and International Application Publication Nos. WO 2001/19800, WO 2001/26644, WO 2001/27135, WO 2001/74344, WO 2003/011219, WO 2003/088970, WO 2004/020599, WO 2005/013800, WO 2005/033288, WO 2005/032343, WO 2005/042700, WO 2006/028958, WO 2006/050351, WO 2006/078283, WO 2007/054623, WO 2007/059157, WO 2007/120827, WO 2007/131201, WO 2008/070357, WO 2008/110611, WO 2008/112913, and WO 2008/131354.

Additional examples of hedgehog inhibitors include, but are not limited to, vitamin D3, itraconazole, GDC-0449 (also known as RG3616 or vismodegib) described in, e.g., Von Hoff D. et al., N. Engl. J. Med. 361(12):1164-72 (2009); Robarge K. D. et al., Bioorg Med Chem Lett., 19(19):5576-81 (2009); Rudin, C. et al., New England J. of Medicine, 361-366 (2009); BMS-833923 (also known as XL139) described in, e.g., in Siu, L. et aL., J. Clin. Oncol. 28:15s (suppl; abstr 2501) (2010); LDE-225 described, e.g., in Pan S. et al., ACS Med. Chem. Lett. 1(3): 130-134 (2010); LEO-506 described, e.g., in National Institute of Health Clinical Trial identifier No. NCT01106508; PP-04449913 described, e.g., in National Institute of Health Clinical Trial Identifier No, NCT00953758; Hedgehog pathway antagonists disclosed in U.S. Patent Application Publication No. 2010/0286114; SMOi2-17 described, e.g., U.S. Patent Application Publication No. 2010/0093625; SANT-1 and SANT-2 described, e.g., in Chen J. K. et al., Proc. Natl. Acad. Sci. USA 99: 14071-14076 (2002) and in Rominger C. M. et al., J. Pharmacol. Exp. Ther., 329(3):995-1005 (2009); 1-piperazinyl-4-arylphthalazines or analogues thereof, described in Lucas B. S. et al., Bioorg. Med. Chem. Lett., 20(12):3618-22 (2010).

In certain embodiments, the hedgehog inhibitor is a compound of formula (I):

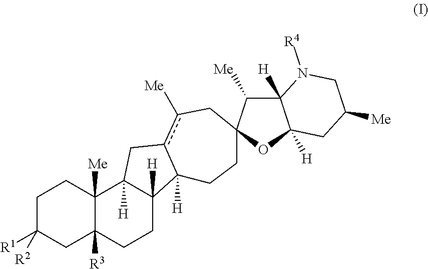

or a pharmaceutically acceptable form thereof (e.g., a salt and/or solvate) thereof; wherein:

R¹ is H, alkyl, —OR, amino, sulfonamido, sulfonamido, —OC(O)R⁵, —N(R⁵)C(O)R⁵, or a sugar;

R² is H, alkyl, alkenyl, alkynyl, aryl, cycloalkyl, nitrile, or heterocycloalkyl; or R¹ and R² taken together form ═O, ═S, ═N(OR), ═N(R), ═N(NR₂), or ═C(R)₂;

R³ is H, alkenyl, or alkynyl;

R⁴ is H, alkyl, alkenyl, alkynyl, aryl, cycloalkyl, heterocycloalkyl, aralkyl, heteroaryl, heteroaralkyl, haloalkyl, —OR, —C(O)R⁵, CO₂R⁵, —SO₂R⁵, —C(O)N(R⁵)(R⁵), —[C(R)₂]_{q}—R⁵, —[(W)—N(R)C(O)]_{q}R⁵, —[(W) CO)]_{q}R⁵, —[(W)—C(O)O]_{q}R⁵, —[(W)—OC(O)]_{q}R⁵, —[(W)—SO₂]_{q}R⁵, —[(W) N(R⁵)SO₂]_{q}R⁵, —[(W) C(O)N(R⁵)]_{q}R⁵, —[(W)—O]_{q}R⁵, —[(W)—N(R)]_{q}R⁵, —W—NR₃ ⁺X⁻or —[(W)—S]_{q}R⁵; wherein each W is independently for each occurrence a diradical such as an alkylene; each q is independently for each occurrence 1, 2, 3, 4, 5 or 6; and X⁻ is an anion (e.g., a halide);

each R⁵ is independently for each occurrence H, alkyl, alkenyl, alkynyl, aryl, heterocycloalkyl, aralkyl, heteroaryl, heteroaralkyl or —[C(R)₂]ₚ—R⁶; wherein p is 0-6; or any two occurrences of R⁵ on the same substituent can be taken together to form a 4-8 membered optionally substituted ring which contains 0-3 heteroatoms selected from N, O, S, and P; and

each R⁶ is independently hydroxyl, —N(R)COR, —N(R)C(O)OR, —N(R)SO₂(R), —C(O)N(R)₂, —OC(O)N(R)(R), —SO₂N(R)(R), —N(R)(R), —COOR, —C(O)N(OH)(R), —OS(O)₂OR, —S(O)₂OR, —OP(O)(OR)(OR), —NP(O)(OR)(OR), or —P(O)(OR)(OR); and

each R is independently H, alkyl, alkenyl, alkynyl, aryl, cycloalkyl or aralkyl; provided that when R², R³ are H and R⁴ is hydroxyl; R¹ cannot be hydroxyl; provided that when R², R³, and R⁴ are H; R¹ cannot be hydroxyl; and provided that when R², R³, and R⁴ are H; R¹ cannot be sugar

In certain embodiments, R¹ is H, hydroxyl, alkoxyl, aryloxy, or amino.

In some embodiments, R¹ and R² taken together along with the carbon to which they are bonded, form ═O, ═N(OR), or ═S.

In other embodiments, R³ is H and/or R⁴ is H, alkyl, hydroxyl, aralkyl, —[C(R)₂]_{q}—R⁵, —[(W)—N(R)C(O)]_{q}R⁵, —[(W)—N(R)SO₂]_{q}R⁵, —[(W)—C(O)N(R)]_{q}R⁵, —[(W)—O)]_{q}R⁵, —[(W)—C(O)]_{q}R⁵, or —[(W)—C(O)O]_{q}R⁵.

In yet other embodiments, R¹ is H or —OR, R² is H or alkyl, and R⁴ is H.

In yet other embodiments, R² is H or alkyl, R³ is H, alkyl, alkenyl, alkynyl, aryl, cycloalkyl, heterocycloalkyl, or aralkyl; and/or R⁴ is H, alkyl, aralkyl, —[(W)—N(R)C(O)]_{q}R⁵, —[(W)—N(R)SO₂]_{q}R⁵, —[(W)—C(O)N(R)]_{q}R⁵, —[(W)—O]_{q}R⁵, —[(W)—C(O)]_{q}R⁵, or —[(W)—C(O)O]_{q}R⁵.

In yet other embodiments, R¹ is sulfonamido.

Specific examples of hedgehog inhibitors include compounds, or pharmaceutically acceptable salts and/or solvates thereof, described in U.S. Pat. No. 7,812,164, incorporated by reference herein. An illustrative example of a hedgehog inhibitor is the following compound, referred to herein as patidegib, previously referred to as “saridegib” and also known in the art as IPI-926:

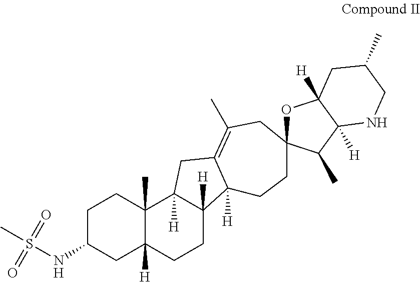

The hedgehog inhibitor compounds described herein can be employed in a pharmaceutically acceptable salt form, and in one embodiment example, the salt form is the hydrochloride salt of Compound II, identified below as Compound II-a:

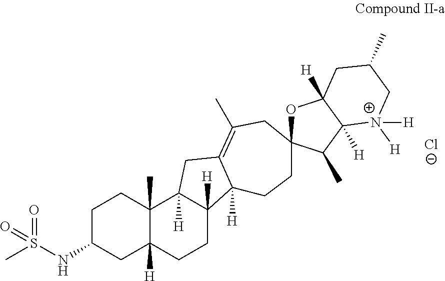

### C. Methods of Treatment

Accordingly, the compositions described hereinabove comprising a hedgehog inhibitor compound are contemplated for use in a method of treating pruritus, where an effective amount of a hedgehog inhibitor compound is topically administered in the form of a topical composition to a subject in need of such treatment. As noted above, reference to a hedgehog inhibitor compound includes the free base thereof, metabolites thereof, derivatives thereof, solvates thereof (e.g., hydrates, alcoholates, etc.) and/or pharmaceutically acceptable salts or esters thereof.

In one embodiment, the method comprises treating pruritus associated with a systemic condition. In various embodiments, the systemic condition is associated with an endocrine or metabolic disease, such as chronic renal failure, diabetes mellitus, hyperthyroidism, hypothyroidism, liver disease, malabsorption or perimenopausal pruritus. In another embodiment, the systemic condition is an infectious (or infestation) disease, such as helminthosis, a viral infection (e.g., an HIV infection, a herpes simplex viral infection, a herpex zoster viral infection), or parasitosis. In another embodiment, the systemic condition is pregnancy and the itching is pruritus gravidarum, with or without cholestasis. In another embodiment, the systemic condition is oral ingestion of a drug that causes skin itching, and exemplary drugs include allopurinol, amiodarone, angiotensin-converting enzyme inhibitors, estrogen, hydrochlorothiazide, opioids, and simvastatin.

In one embodiment, the method comprises treating pruritus associated with a dermatologic disorder. In various embodiments, the dermatologic disorder is an autoimmune disorder (dermatitis herpetiformis, dermatomyositis, pemphigoid, Sjogren's syndrome), a genetic disorder (Darter's disease, Hailey-Hailey disease, ichthyoses, Sjogren-Larsson syndrome), an infection or infestation (arthropod reactions, dermatophytosis, folliculitis, impetigo and other bacterial infections, insect bites, pediculosis, scabies, viral), or an inflammatory condition (asteatosis, atopic eczema, contact dermatitis, drug reactions, invisible dermatoses, lichen planus, lichen simplex chronicus, mastocytosis, miliaria, psoriasis, scars, urticaria)

Pruritus includes any itchy or pruritic condition, e.g., a sensation that causes the desire or reflex to scratch. In some embodiments, the method of treatment described herein is contemplated for the treatment of a subject suffering from a pruritic condition selected from the group consisting of atopic dermatitis, nervous dermatitis, contact dermatitis, seborrheic dermatitis, autosensitization dermatitis, caterpillar dermatitis, asteatosis, senile pruritus cutaneous, insect sting, photosensitive dermatosis, urticarial, prurigo, herpes, impetigo, eczema, tinea, lichen, psoriasis, scabies and acne vulgaris, visceral diseases complicated with pruritus such as malignant tumors, diabetes mellitus, hepatic diseases, renal failure, hemodialysis, peritoneal dialysis, and pregnancy.

In some embodiments, methods are used for the treatment of a subject suffering from a pruritic condition associated with a skin change is contemplated. For example, such pruritic condition can be selected from the group consisting of pruritus secondary to inflamed skin (e.g., atopic dermatitis, psoriasis, burns); pruritus arising from conditions of non-diseased skin (e.g., uremic pruritus, cholestatic pruritus, cancers, hydroxyethyl starch induced pruritus), and pruritus associated with chronic secondary scratch or other types of skin lesions that may or may not be the result of an underlying medical condition (e.g., prurigo nodularis) and the underlying disease is categorized based on histological, radiological or other investigations as being of an origin selected from the group consisting of dermatologic origin, systemic disease origin, neurologic origin, psychogenic origin, mixed origin, or other origin.

Depending on the nature of the composition, it may be topically applied directly to the skin, for example, by rubbing with the fingertips of a subject in need or by a caregiver, or for example, by spraying the solution or suspension onto the skin. The composition may be topically applied to the skin at the site of itch, or may be topically applied to the skin in an area peripheral or adjacent the site of itch.

The dosing frequency will vary according to the subject and the underlying cause of itch. Application of the composition less than once per day or multiple times per day is contemplated. In one embodiment, the composition is topically applied to the skin periodically less than once per day. In other embodiments, the composition is topically applied to the skin periodically from 1-8 times per day.

The effectiveness of the treatment or of a selected dosage regimen can be determined by evaluation via a Pruritus Visual Analog Scale (VAS) test. The effectiveness of a dosage regimen can be determined by evaluation via a Numerical Rating Scale (NRS). The effectiveness of a dosage regimen can be determined by evaluation via a Numerical Rating Scale (NRS) in association with Medical Outcomes Study (MOS), Itch MOS Sleep scale, Hospital Anxiety and Depression Scale (HADS), Patient Assessed Disease Severity Scale, or Skindex-10 or a combination thereof. The effectiveness of a dosage regimen can also be determined by evaluation via a NRS independently or in association with Patient Global Assessment (PGA) via ItchApp, vPGA, Dermatology Life Quality Index (DLQI), Patient Benefit Index (PBI), MOS Sleep Scale, or HADS or a combination thereof. In still yet another embodiment, the effectiveness of a dosage regimen can be determined by evaluation via a NRS independently or in association with Prurigo Activity Score (PAS), Nocturnal scratching using actigraphy, nerve fiber density and MOR/KOR density.

In other embodiments, determining the effectiveness of the dose of a hedgehog inhibitor compound in the topically applied composition or determining the effective dosing regimen can include evaluation of the patient's pruritus symptoms using an instrument selected from the group consisting of Pruritus Visual Analog Scale (VAS) test, Brief Itching Inventory, Skindex-10, Itch MOS of sleep, Beck Depression Index, and Patient Categorization of Pruritus Disease Severity.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A method for treating pruritus in a mammalian subject, comprising:
topically applying a composition comprising a hedgehog inhibitor in an amount effective for treatment of pruritus.

2. The method of claim 1, wherein said composition comprises a liquid, a semi-solid or a solid composition.

3. The method of claim 2, wherein the composition is a foam.

4. The method of claim 2, wherein the composition is a liquid in the form of a lotion.

5. The method of claim 1, wherein said composition is topically applied to the skin at the site of itch.

6. The method claim 1, wherein the composition is a semi-solid or solid composition and the method further comprises rubbing the composition into the skin.

7. The method of claim 1, wherein said composition is topically applied to the skin periodically less than once per day.

8. The method of claim 1, wherein said composition is topically applied to the skin periodically from 1-8 times per day.

9. The method of claim 7, wherein the composition is applied to the site of an itch.

10. The method of claim 1, wherein the composition comprises between about 0.01-10 percent by weight of the hedgehog inhibitor.

11. The method of claim 10, wherein the hedgehog inhibitor is patidegib.

12. The method of claim 1, wherein said pruritus is associated with a pruritic condition selected from atopic dermatitis, nervous dermatitis, contact dermatitis, seborrheic dermatitis, autosensitization dermatitis, caterpillar dermatitis, asteatosis, senile pruritus, insect sting or bite, photosensitive dermatosis, urticaria, prurigo, herpes, impetigo, eczema, tinea, lichen, psoriasis, scabies and acne vulgaris.

13. The method of claim 1, wherein said pruritus is associated with an underlying medical condition.

14. The method of claim 13, wherein the underlying medical condition has an origin comprising dermatologic origin, systemic disease origin, neurologic origin, or psychogenic origin.

15. The method of claim 1, wherein the subject has uremic pruritus or prurigo nodularis.

16. The method of claim 1, wherein the subject has pruritus secondary to a systemic condition selected from an endocrine or metabolic disease, an infectious disease, pregnancy, or a drug.

17. The method of claim 16, wherein the endocrine or metabolic disease is selected from chronic renal failure, diabetes mellitus, hyperthyroidism, hypothyroidism, liver disease, malabsorption and perimenopausal pruritus.

18. The method of claim 16, wherein the infectious disease is selected from helminthosis, a viral infection, and parasitosis.

19. The method of claim 16, wherein the subject is pregnant and suffers from pruritus gravidarum with or without cholestasis.

20. A method for relieving itching on mammalian skin of a subject, comprising:
topically applying a composition comprising a hedgehog inhibitor compound in an amount effective to relieve itching.

21. The method of claim 20, wherein said composition comprises a liquid, a semi-solid or a solid composition.

22. The method of claim 20, wherein said composition is topically applied to the skin at the site of itch.

23. The method of claim 20, wherein the composition is a semi-solid or solid composition and the method further comprises rubbing the composition into the skin.

24. The method of claim 20, wherein the composition comprises between about 0.01-10 percent by weight of the hedgehog inhibitor.

25. The method of claim 24, wherein the hedgehog inhibitor is patidegib.
